(19) United States
(12) Patent Application Publication
(10) Pub. No.: US 2011/0054438 A1
(43) Pub. Date: Mar. 3, 2011

(54) STENT DELIVERY AT A BIFURCATION, SYSTEMS AND METHODS

(76) Inventors: Mark W.I. Webster, Auckland (NZ); John A. Ormiston, Auckland (NZ)

(21) Appl. No.: 12/549,969

(22) Filed: Aug. 28, 2009

(51) Int. Cl. A61M 31/00; A61M 29/00; A61F 2/82

(52) U.S. Cl. 604/500; 606/194; 604/96.01; 623/1.23

(57) ABSTRACT

The present invention provides systems and methods for delivery of a stent or scaffold at a blood vessel or body lumen bifurcation. A method can include positioning a scaffold at a bifurcation of a main lumen into first and second branch lumens using a first expansion catheter, inflating a main balloon of the catheter to expand the proximal portion of the scaffold, positioning a second expansion system, and expanding the distal portion of the scaffold on a generally distal side of the bifurcation.

### BACKGROUND OF THE INVENTION

The present invention relates generally to intravascular catheter devices and stents. More particularly, the present invention describes catheters, systems and assemblies, as well as methods for delivery of stents or scaffolds at bifurcations, e.g., for repairing diseased blood vessels at a bifurcation.

Intravascular stents or scaffolds are used for various purposes, including opening occluded blood vessels. In a typical approach, a stent is provided in a contracted form, e.g., disposed surrounding a deflated balloon positioned inside the stent. The stent and balloon are positioned at a distal portion of a catheter device, and a physician or operator inserts a guidewire into the lumen or blood vessel, and then slides the catheter over the wire to position the stent in the desired location. The stent is then expanded, e.g., via balloon expansion, so that the stent is anchored in place to hold the vessel open. Once the stent has been expanded, the balloon is deflated and the catheter withdrawn from the blood vessel. Treatment can also be combined with therapeutic agents or pharmaceuticals to improve healing and/or prevent restenosis.

Repairing blood vessels at a bifurcation has been particularly challenging for several reasons, including the need for an accurate and deliberate positioning of the stent in a precise location that provides adequate coverage of the diseased area, but while maintaining blood vessel patency and allowing adequate blood flow through the vessels at or downstream of the treatment site. Prior treatment using conventional tubular or cylindrical stents has presented a variety of problems. For example, conventional stents have been deployed so that either the entire stent is in the main lumen upstream of the bifurcation, or the proximal portion of the stent is positioned in the main lumen upstream of the bifurcation, while the distal portion is located entirely or unevenly in a downstream branch vessel. In the former case, the stent is often not advanced distally enough to provide the desired coverage of the diseased area. In the latter instance, vessel repair often comes at the expense of obstructing or failing one of the branch lumens or downstream vessels. Another approach has included positioning of multiple different stents at different locations in the bifurcation. Besides conventional cylindrical stents, more elaborate designs, such as bifurcated stents, have become available. Use of such non-conventional stents, however, like the use of multiple conventional stents, undesirably increases the complexity and invasiveness of the treatment.

Thus, a need exists for improved methods and systems for delivery of stents or scaffolds at bifurcations, thereby providing more efficient and effective repair of diseased blood vessels at a bifurcation.

### BRIEF SUMMARY OF THE INVENTION

The present invention provides systems and methods for delivery of a stent or scaffold at a body lumen or blood vessel bifurcation in a patient and, more particularly, repairing diseased blood vessels at a bifurcation. A system includes a first scaffold deployment catheter with at least one balloon located on a distal portion of the catheter. A non-bifurcated or standard tubular scaffold or stent is used, and is placed so that a proximal portion of the scaffold is over a balloon and a distal portion is over a balloon or extends distal to a balloon. The catheter includes one or more guidewire lumens, and the catheter can be delivered over one or more guidewires so that the end of the catheter enters into the bifurcation and the distal end of the scaffold diverges or spreads over the carina. The balloon(s) may then be expanded to open and anchor the proximal portion of the scaffold. The first catheter is then withdrawn and a second expansion system, such as a pair of kissing balloons or single bifurcated balloon, is advanced into position and expanded to deploy the distal portion of the scaffold.

The systems and methods of the present invention may provide numerous advantages, including a more simplified system using a standard tubular scaffold or stent, compared to approaches requiring delivery of numerous stents, advancing guidewires though the struts of delivered stents, or use of stents of more elaborate design, such as bifurcated stents. The present techniques further provide for advancement of the distal portion of the scaffold deeper into the bifurcation so that the delivered scaffold covers the outer flank of the bifurcation where the diseased portion is most prevalent, while the inner carina is usually open and does not need scaffold coverage. Guidewires are maintained in both branch lumens throughout the stent delivery procedure.

Thus, in one aspect, the present invention includes systems and methods for positioning a cylindrical scaffold at a bifurcation of a main lumen into first and second branch lumens. A method can include positioning a scaffold at a bifurcation of a main lumen into first and second branch lumens, including advancing a first balloon catheter system to the bifurcation. The first catheter system can include at least one main balloon with a scaffold positioned over the main balloon(s), or partly over the main balloon(s) and partly distal to the main balloon(s). The main balloon catheter system is advanced until the distal portion of the scaffold has entered the bifurcation, and the main balloon(s) is/are inflated to expand the proximal portion of the scaffold on a generally proximal side of the bifurcation while the distal portion of the scaffold remains partially or substantially unexpanded. Further, a second scaffold expansion system is introduced, e.g., following main catheter withdrawal, and expanded or balloon inflated to expand the distal portion of the scaffold on a generally distal side of the bifurcation. Thus, the proximal portion of the scaffold and the distal portion of the scaffold can be expanded in seriatim.

In another aspect, a method includes placing a first guidewire from the main lumen into the first branch lumen and a second guidewire from the main lumen into the second branch lumen. Following positioning of the first and second guidewires, a main balloon catheter is advanced over at least one guidewire. The scaffold can be coupled with the main balloon catheter such that a proximal portion of the scaffold is positioned over a balloon and a distal portion of the scaffold is positioned over a balloon or distally of a balloon. The balloon catheter is advanced until the distal portion of the scaffold has entered the bifurcation. Following positioning the scaffold in the bifurcation, the main balloon(s) is/are inflated to expand the proximal portion of the scaffold on a generally proximal side of the bifurcation while the distal portion of the scaffold remains partially or substantially unexpanded. The main balloon is removed from the bifurcation, and first and second balloon catheters are introduced over a first guidewire from the main lumen into the first branch lumen and over a second guidewire from the main lumen into the second branch lumen, respectively, so that said first and second balloons are positioned, e.g. side-by-side, within the distal portion of the scaffold. Following introduction of the first and second balloons, the balloons are inflated to expand the distal portion of the scaffold on a generally distal side of the bifurcation.

In another aspect, the present invention includes scaffold delivery catheters and systems. A catheter can include a catheter body having a distal end, a proximal end, and at least one guidewire lumen extending to the distal end, at least one main inflatable balloon disposed on the catheter body at a predetermined distance proximally of the distal end. The catheter can further include a cylindrical scaffold having a proximal portion disposed over an inflatable balloon and a distal portion disposed over a balloon or distally of an inflatable balloon. The scaffold can be disposed over a balloon such that inflation of one or more inflatable balloon(s) will expand the proximal portion while leaving the distal portion of the scaffold partially or substantially unexpanded. Guidewire assemblies suitable for use with catheters and techniques described herein are further provided.

For a fuller understanding of the nature and advantages of the present invention, reference should be made to the ensuing detailed description and accompanying drawings. Other aspects, objects and advantages of the invention will be apparent from the drawings and detailed description that follows.

### BRIEF DESCRIPTION OF THE DRAWINGS

FIGS. 1A through 1C provide a simplified illustration of scaffold positioning at a bifurcation including a main vessel lumen and branch lumens, according to an embodiment of the present invention.

FIG. 2 illustrates a balloon catheter with a bifurcated distal portion and a scaffold positioned on the catheter, according to an embodiment of the present invention.

FIGS. 3A through 3D illustrate scaffold positioning at a bifurcation, according to an embodiment of the present invention.

FIGS. 4A through 4F illustrate scaffold positioning at a bifurcation, according to yet another embodiment of the present invention.

FIG. 5 illustrates a balloon catheter having a non-bifurcated distal portion and a scaffold positioned on the catheter, according to an embodiment of the present invention.

FIG. 6 shows a balloon catheter having a bifurcated distal portion and a scaffold positioned on the catheter, and further having a first and second guidewire coupled with the catheter, according to an embodiment of the present invention.

FIGS. 7A and 7B illustrate embodiments of balloon catheters having a bifurcated distal portion, according to embodiments of the present invention. FIG. 7A shows a catheter having a balloon encircling both first and second guidewire lumens. FIG. 7B shows a catheter having a balloon encircling a first guidewire lumen with a second guidewire lumen disposed opposite an outer surface of the balloon.

FIGS. 8A and 8B illustrate a balloon catheter having a scaffold positioned over a balloon, the catheter coupled with a guidewire assembly and further coupled with a retractable sheath, in accordance with further embodiments of the present invention.

FIGS. 9A and 9B illustrate a balloon catheter having a scaffold positioned over a balloon, where the balloon is configured for a rolling-type movement along a longitudinal length of the catheter.

FIGS. 10A and 10B illustrate an exemplary guidewire assembly according to an embodiment of the present invention, including a first guidewire having a lumen and a second guidewire sized for positioning and advancement through the first guidewire lumen.

FIGS. 11A and 11B illustrate a guidewire assembly according to an exemplary embodiment of the present invention, the assembly including a first guidewire having a lumen extending distally to a side opening and a second guidewire sized for positioning and advancement in the lumen.

FIGS. 12A and 12B illustrate yet another guidewire assembly according to an exemplary embodiment of the present invention, the assembly including guidewires having a generally cylindrical distal portion and a proximal portion having a “D-shaped” cross-sectional geometry.

FIGS. 13A and 13B illustrate a balloon catheter having a scaffold positioned over a balloon, the balloon encircling one of two guidewire lumens, and the inflated diameter of the proximal portion of the balloon greater than the inflated diameter of the distal portion of the balloon

FIGS. 14A and 14B illustrate a balloon catheter having a scaffold positioned over two balloons on two guidewire lumens. The proximal balloon has a larger inflated diameter than the distal balloon.

FIGS. 15A and 15B illustrate a balloon catheter having a scaffold positioned over a balloon and two guidewire lumens, whereby the balloon catheter and scaffold is advanced into the vessel and close to the bifurcation in part over one guidewire lumen and in part over the other guidewire lumen

### DETAILED DESCRIPTION OF THE INVENTION

The present invention provides systems and methods for delivery of a stent or scaffold at a blood vessel or body lumen bifurcation, e.g., for repairing diseased blood vessels at a bifurcation in a patient. A bifurcation can include, e.g., a region of a vessel or lumen at or near a point where division of two or more branches occurs. A bifurcation as referred to herein can include a generally symmetric or, more typically, generally asymmetric bifurcation. The systems and methods of the present invention deliver a staged deployment or expansion of a tubular scaffold or stent, or stent/scaffold generally non-bifurcated in having an end with a single opening rather than a plurality of different openings. According to the present invention, a scaffold can be more optimally advanced and positioned into the bifurcation such that the delivered scaffold covers the outer flank of the bifurcation where the diseased portion may be most prevalent.

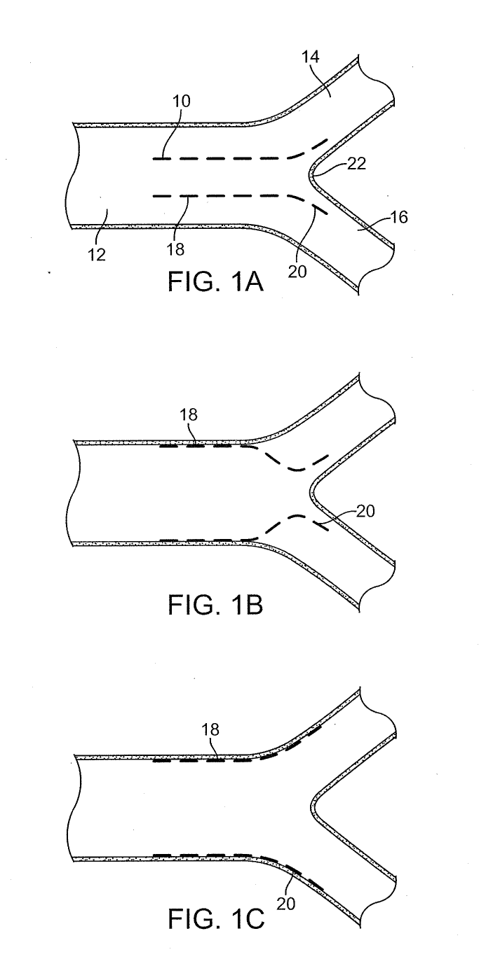

FIGS. 1A through 1C include a simplified side view illustrating general aspects of scaffold positioning at a bifurcation according to an embodiment of the present invention. The scaffold and positioning thereof is shown sans catheters for clarity. Non-limiting, exemplary catheter systems are described further below. Scaffold 10 is advanced, e.g., over an expandable balloon catheter system, through the vasculature of a patient and is aligned adjacent to or approaches the bifurcation of a main lumen 12 into branch lumens 14 and 16. The scaffold 10 includes a simple cylindrical or tubular shaped scaffold or stent, including a proximal portion 18 and a distal portion 20. The scaffold 10 is entered into the bifurcation (e.g., aligned adjacent to or approaches the bifurcation), and may be positioned such that the distal end of the scaffold diverges or spreads over the carina 22, and at least partially into one or more branch lumens. The scaffold 10 is then expanded and positioned, or anchored, to the lumen wall in a staged manner. Expansion and scaffold positioning includes a first expansion where a proximal portion 18 of the scaffold is deployed (FIG. 1B), followed by expansion or deployment of a distal portion 20 of the scaffold (FIG. 1C).

The terms “stent” and “scaffold” are generally used interchangeably herein, and include any of the array of expandable (e.g., balloon expandable) prostheses and scaffolds which are introduced into a lumen at a target treatment site and expanded in situ to exert a radially outward force against the lumen wall for positioning or anchoring in the lumen wall at the treatment site. Any number of scaffold or stent configurations may be employed in the systems of the present invention. The scaffolds or stents of the present invention can include a closed or an open lattice structure, and will typically be fabricated from a malleable or elastic material. Malleable materials such as stainless steel, gold, platinum, titanium, cobalt chromium and other alloys or bioabsorbable materials such as polymers may be selected for stent composition, and scaffolds are typically expanded by balloon inflation, causing deformation of the lattice so that it remains deformed in an open position after deployment. Particular patterns, such as ring and link patterns, element dimensions and material composition can be selected to accommodate a particular application including, but not limited to, variations in length, expanded diameter, coverage, flexibility, resorbability, and/or the like. Scaffolds will typically include a length of about 6 mm to about 38 mm. As described further herein, scaffolds can be disposed on a balloon of a catheter such that the proximal portion of the scaffold is positioned on a balloon with the distal portion of the scaffold is either on a balloon or extends distally to a balloon. In the embodiments with a distal portion of the scaffold extending distally to a balloon, the portion of a cylindrical scaffold disposed distally of an inflated balloon will typically have a length in the range of about 2 mm to about 12 mm. Scaffolds may also be coated with one or more compositions such as a therapeutic agent or drug (e.g., anti-restenosis drug), including those known in the art. Deployment of a scaffold is typically described herein in the context of a coronary artery stent procedure, but it should be understood that the invention may be employed in any variety of blood vessels and other body lumens in which stents or tubular scaffolds are deployed.

Various catheter systems, scaffold delivery catheters, including balloon catheters, and designs may be employed for scaffold delivery as described herein, and non-limiting exemplary systems and designs are described further herein. Catheter systems can be configured in a conventional manner in the sense that they can be configured for use with one or more guidewires, and will typically include an internal guidewire lumen for receiving a guidewire. Deployment catheters and catheter systems of the present invention can rely on any of a number of guidewire configurations, including rapid exchange, over-the-wire, or a combination of both. While certain exemplary embodiments described herein may be illustrated with a particular guidewire configuration, it will be recognized that additional guidewire configurations are included within the scope of the present invention. Additionally, balloon catheters as described herein will include an inflation lumen through which the balloon is inflated and deflated. Catheter balloons can vary in design or sizing, and may be selected at least partially on the intended use and/or treatment requirements. Catheter balloons, for example, will typically range from about 4 mm to about 26 mm in length. Catheters may further include one or more markers, such as radiopaque markers, to facilitate tracking of the device during an interventional procedure.

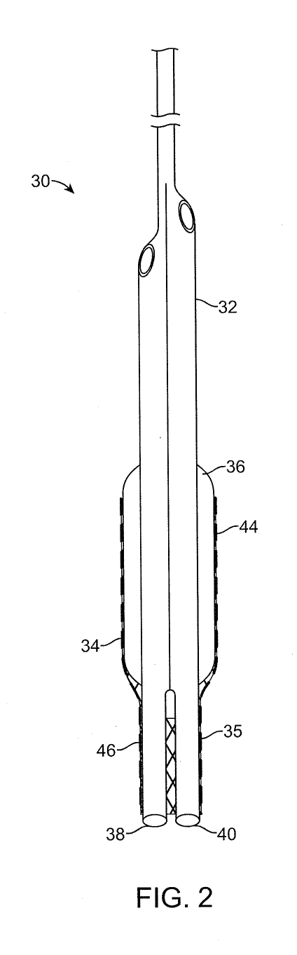

A bifurcation scaffold delivery system according to an embodiment of the present invention is illustrated with reference to FIG. 2. The catheter system 30 includes a first scaffold deployment catheter 32, configured for initial positioning of the scaffold 34 at a desired location in a bifurcation at a first stage, where the scaffold 34 is advanced to a bifurcation and the proximal portion of the scaffold is expanded. The catheter 32 includes a bifurcated distal portion 35 and a balloon 36 located proximally of the bifurcation 35. Each of the bifurcations include a guidewire lumen 38, 40. A non-bifurcated scaffold (e.g., standard tubular stent) is positioned on the catheter balloon 36, the scaffold 34 including a proximal portion 44 and a distal portion 46. The scaffold 34 is disposed such that the proximal portion 44 is positioned over the balloon 36 and the distal portion 46 is positioned distally of the balloon 36 and over the bifurcation 35.

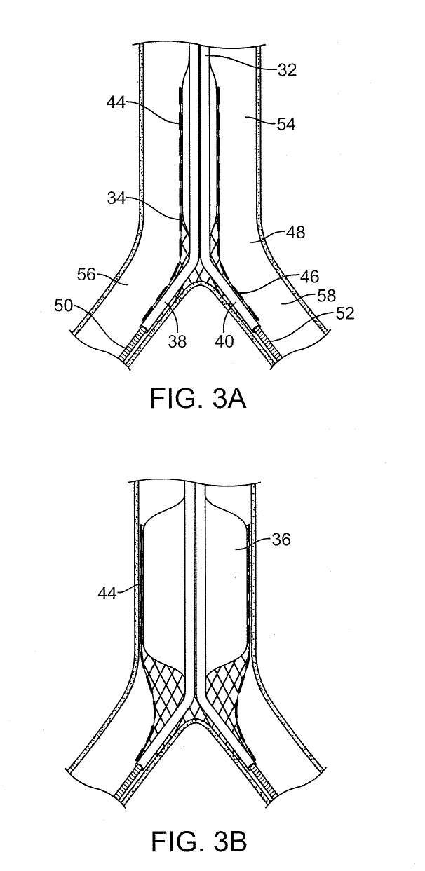

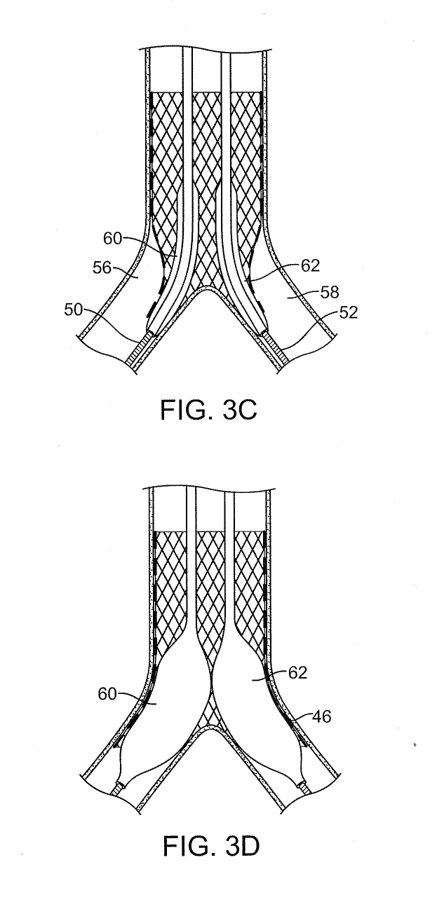

Positioning a scaffold within a bifurcation using a system similar to the one described in FIG. 2 above, is described with reference to FIGS. 3A through 3D. The system can be advanced to the bifurcation 48 or treatment site via advancement over one or more guidewires 50, 52 positioned in the lumen(s). As described above, the system includes a scaffold deployment catheter 32, designed for advancement through the main lumen 54 and into the bifurcation and branch lumens 56, 58. The catheter includes a main balloon 36 located proximally of a bifurcated distal end. The distal end includes guidewire lumens 38, 40. During at least initial positioning, one or more guidewires 50, 52 can be advanced through the main lumen and into one or more of the branch lumens. Where a dual guidewire configuration is used, a first guidewire 50 is advanced through the main lumen and into branch lumen 56, and a second guidewire 52 is similarly advanced through the main lumen and into branch lumen 58. The guidewires 50, 52 can be advanced and positioned in seriatim or substantially at the same time. With one or more guidewires positioned in the bifurcation, the catheter 32 can be advanced over one of the guidewires 50, 52, or both. For example, in one embodiment, both guidewires 50, 52 are advanced and positioned in branch lumens 56, 58, respectively, and the catheter 32 is loaded over such that the guidewires 50, 52 are run through lumens 38, 40, respectively.

In another embodiment, a first guidewire is advanced into a branch lumen and the catheter is loaded over the guidewire and advanced to the bifurcation, while a second guidewire can be loaded into the catheter prior to advancement and positioning and advanced along with the catheter (e.g., similar to examples described further below). In yet another embodiment, the catheter is advanced over a first positioned guidewire, and once the catheter is positioned at or near the bifurcation, a second guidewire can be advanced through a lumen of the catheter and then further into the second branch lumen. Various embodiments of guidewire use and positioning can be utilized in conjunction with scaffold positioning according to the present invention, and certain such embodiments are described further herein.

FIG. 3A illustrates initial positioning of the catheter and scaffold system within the bifurcation. With guidewires 50, 52 running through the main lumen 54 and into branch lumens 56, 58, respectively, distal advancement of the catheter sends guidewire lumens 38, 40 of the catheter 32 into respective branch lumens of the bifurcation. As illustrated, with distal advancement of the catheter, branch lumens 38, 40 diverge slightly or flare out over the carina and permit advancement of the scaffold 34 such that distal portion of the scaffold is advantageously positioned deeper within the bifurcation. Next, the balloon 36 of the catheter is inflated so as to deploy the proximal portion 44 of the scaffold in the vessel or lumen immediately upstream or proximal of the bifurcation (FIG. 3B). The catheter 32 can then be withdrawn and a second deployment system including balloons 60, 62 can be advanced over guidewires 50, 52 and positioned in the bifurcation and at least partially into lumens 56, 58, as shown in FIG. 3C. Balloons 60, 62, can be generally short (e.g., about 4 mm to about 10 mm in length) balloons, with inflated diameter selected or matched relative to the diameter of each downstream vessel or lumen. Balloons 60, 62 can be advanced substantially simultaneously into position, or one at a time. Once in position, balloons 60, 62 can be inflated (e.g., simultaneously inflated) to deploy the distal portion 46 of the scaffold (FIG. 3D). Thus, as illustrated, the scaffold is positioned and then deployed in a staged manner, with the proximal portion of the scaffold (e.g., generally the proximal about ½ to about ¾ portion of the scaffold) deployed by inflation of balloon 36 of the catheter, and then the distal portion of the scaffold (e.g., the distal approximately ¼ to about ½ portion of the scaffold) is deployed via inflation of balloons 60, 62, as illustrated above.

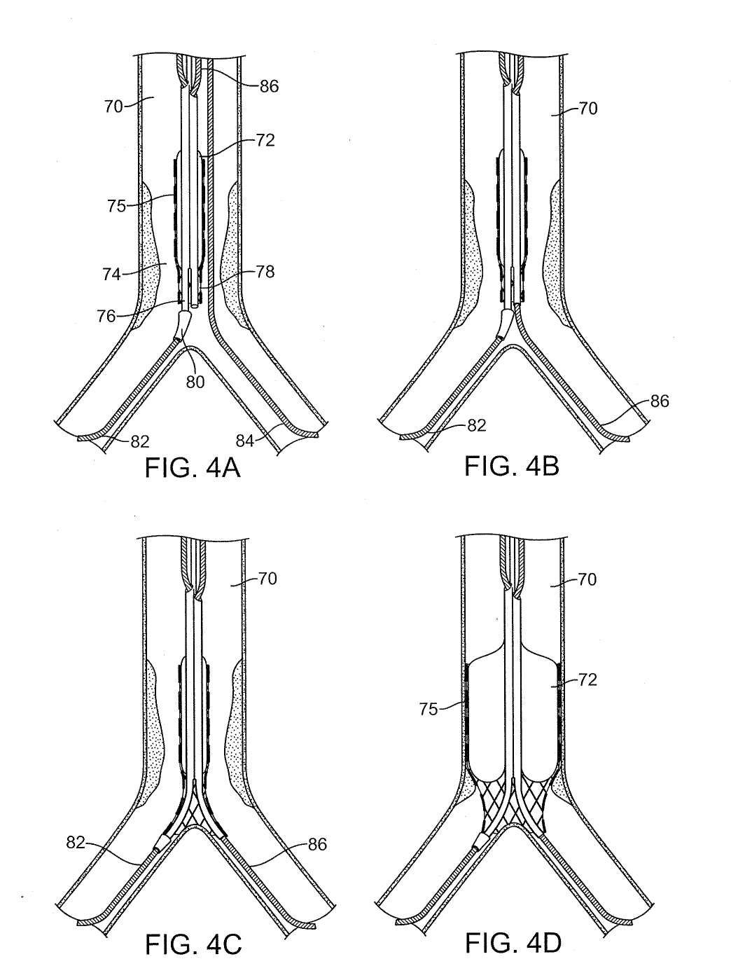

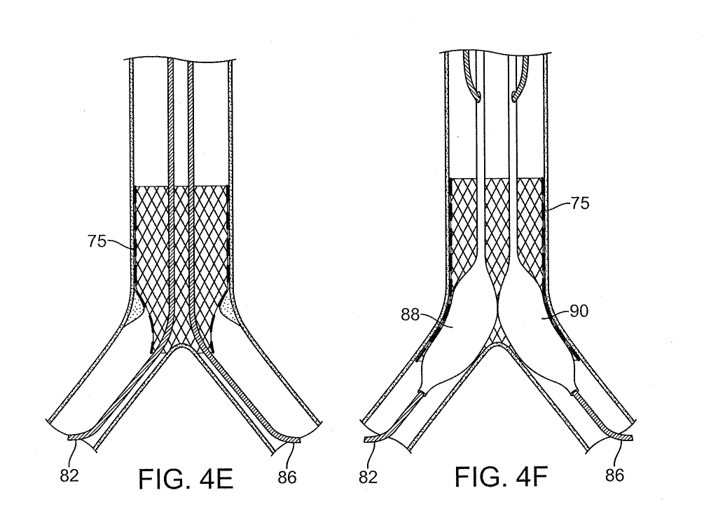

As discussed above, a catheter system of the present invention can be advanced to a bifurcation over a single guidewire configuration or more than one guidewire. In one embodiment, a two-wire system can be utilized, with the scaffold delivery system advanced over a first guidewire with a second wire “loaded” on the catheter. Referring now to FIG. 4A, a two-wire catheter 70 or scaffold delivery system is advanced in a body lumen including a main lumen and branch lumens. The catheter 70 includes a main balloon 72 located proximally of a bifurcated distal end 74. A non-bifurcated scaffold 75 or stent (e.g., standard tubular stent) is positioned on the catheter, the scaffold including a proximal portion and a distal portion, with the proximal portion positioned over the balloon and the distal portion positioned distally of the balloon. The catheter further includes guidewire lumens 76, 78, with the distal portion of guidewire lumen 76 including a tapered nose-cone, which can be shaped and/or composed of a material (e.g., soft elastomeric material) selected to minimize trauma to the vessel during advancement of the catheter. Guidewires 82 and 84 are positioned to extend through the main lumen and into respective branch lumens. Guidewires 82 and 84 maintain branch vessel access and may be used for pre-dilation of the bifurcation with standard balloon catheters, prior to advancing the scaffold delivery system 70 into the vessel. The catheter is advanced over guidewire 82, which runs through lumen 76, while a second guidewire 86 is “loaded” on the catheter, or positioned in lumen 78 without extending substantially out the distal end of lumen 78, as shown in FIG. 4A. Once the delivery system is advanced to a location just upstream of the bifurcation, the loaded wire 86 can be advanced into a downstream branch (FIG. 4B). Once guidewire 86 is advanced into a downstream branch, guidewire 84 can be removed. With guidewires 82 and 86 advanced in branch lumens, the delivery catheter can then be advanced further into the bifurcation (e.g., a final few mm) so that the distal portion of the scaffold enters into the bifurcation, and may be partly flared or splayed on the wires 82, 86, as shown in FIG. 4C. Balloon 72 is expanded so as to deploy the proximal portion of the scaffold 75 (FIG. 4D). The catheter 70 is then withdrawn, leaving guidewires 82 and 86 in position (FIG. 4E). Balloons 88 and 90 are then advanced over wires 82 and 86, and into respective branch lumens, for inflation and deployment of the distal portion of the scaffold 75 (FIG. 4F).

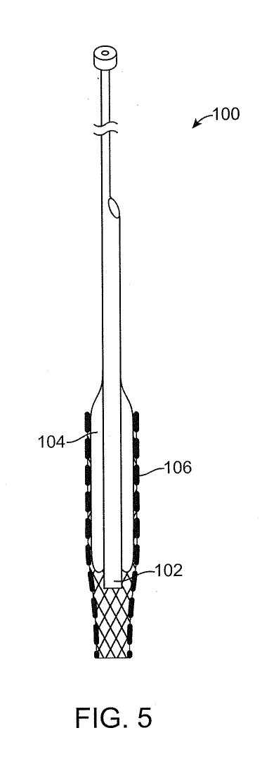

A scaffold delivery system according to another embodiment of the present invention is illustrated with reference to FIG. 5. The system includes a scaffold deployment catheter 100 including a proximal portion and a distal portion. The distal portion includes a guidewire lumen 102 extending through the catheter elongated body and a main balloon 104. The guidewire lumen 102 in the illustrated embodiment includes a non-bifurcated distal portion that is more truncated in comparison to other embodiments described herein. While the catheter is illustrated as having a single guidewire lumen, alternate embodiments can include more than one guidewire lumen. The guidewire lumen can be sized to accommodate a single guidewire, or multiple guidewires, or one or more guidewires of a variety of sizes. The system further includes a tubular or non-bifurcated scaffold 106 positioned on the catheter. The scaffold 106 includes a proximal portion and a distal portion, with the scaffold 106 disposed on the catheter such that the proximal portion is over the balloon 104 and the distal portion is positioned distally of the balloon 104. The catheter 100 can be advanced over one or more guidewires, including, for example, over a guidewire having a bifurcated portion, for positioning of the scaffold at a lumen bifurcation as described further herein.

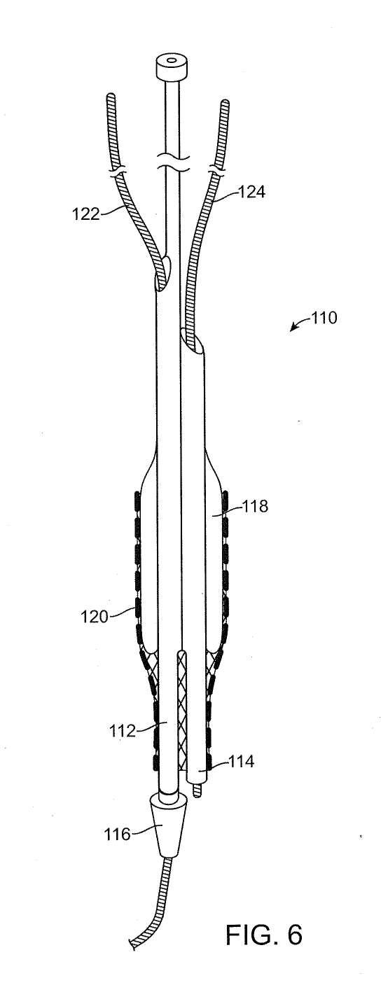

FIG. 6 shows a scaffold delivery system or catheter system according to an embodiment of the present invention, and illustrates a coupling of the delivery system with guidewires where the catheter can be advanced over a first guidewire, with a second guidewire loaded on the catheter. The system includes a catheter 110 having a proximal portion and a distal portion, the distal portion having a bifurcation with lumens 112 and 114 extending along a length of the catheter body and forming a bifurcated distal portion. Lumen 112 further includes a tapered nose-cone 116, and extends more distally relative to lumen 114. The system further includes a balloon 118 disposed on the body of the catheter 110, and scaffold 120 having a proximal portion and distal portion. In use, the first guidewire 122 can be advanced through a body lumen of the patient and positioned at a desired location with respect to a bifurcation or treatment site. The catheter is then advanced over the guidewire and into position. While being advanced, the second guidewire 124 can be positioned loaded onto the catheter and disposed at least partially in lumen 114. As the catheter is advanced over the first guidewire 122 or as the catheter is positioned at a desired location, the loaded second guidewire 124 can be advanced distally and into a branch lumen of the bifurcation. Use of the catheter illustrated in FIG. 6 can be further understood with reference to FIGS. 4A through 4F, where similar catheter positioning is described.

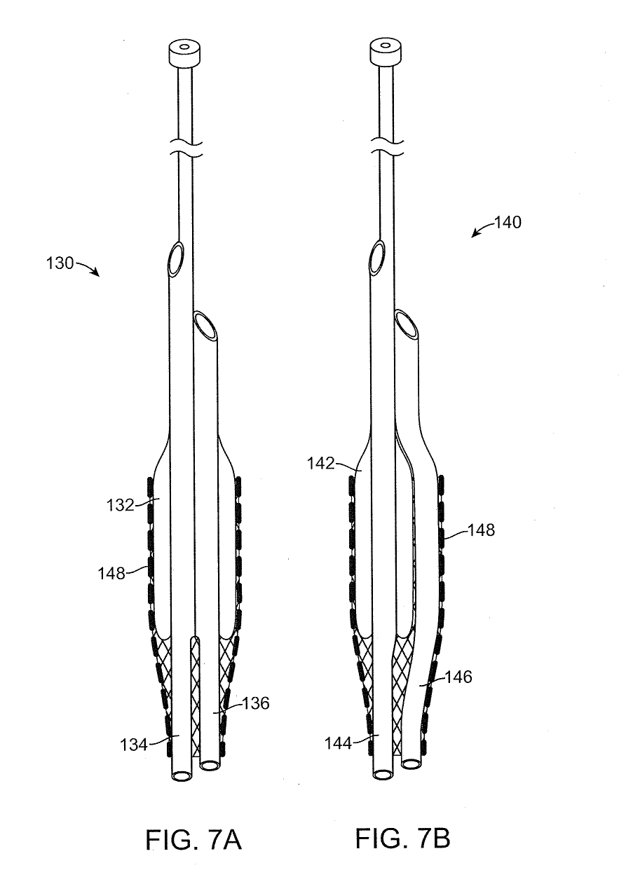

FIGS. 7A and 7B show scaffold delivery systems according to further embodiments of the present invention, illustrating exemplary balloon and lumen configurations. FIG. 7A shows a system including delivery catheter 130 and a scaffold 148 positioned as described elsewhere herein, with a proximal portion of the scaffold disposed over balloon 132, and distal portion of the scaffold positioned distal to the balloon. The catheter 130 includes guidewire lumens 134 and 136, with the inflatable or expandable balloon 132 encircling both lumens 134 and 136. In alternate embodiments, a catheter can be configured such that one or more guidewire lumens is disposed outside an expandable balloon or, in other words, with the one or more guidewire lumens disposed between an outer surface of a balloon and a scaffold positioned on the catheter. FIG. 7B illustrates a scaffold delivery system including a delivery catheter 140 having a balloon 142 encircling a first lumen 144, with a second lumen 146 not encircled or positioned outside the balloon 142. A scaffold 148 is shown coupled with the catheter 140 in a manner as describe elsewhere herein. Guidewire lumen 144 is encircled by balloon 142, whereas guidewire lumen 146 runs outside balloon 142 and is disposed between an outer surface of the balloon 142 and an inner surface of scaffold 148 as shown. Upon expansion/inflation of the balloon 142, lumen 146 and a proximal portion of scaffold 148 are expanded outward. Additional embodiments making use of the concepts described herein may be envisioned and are further included here. For example, in yet another embodiment, a delivery catheter may include both a first and second lumen disposed outside a balloon, such that inflation of the balloon provides lateral movement and expansion of the lumens, as well as a scaffold, disposed outside the balloon in the manner described herein.

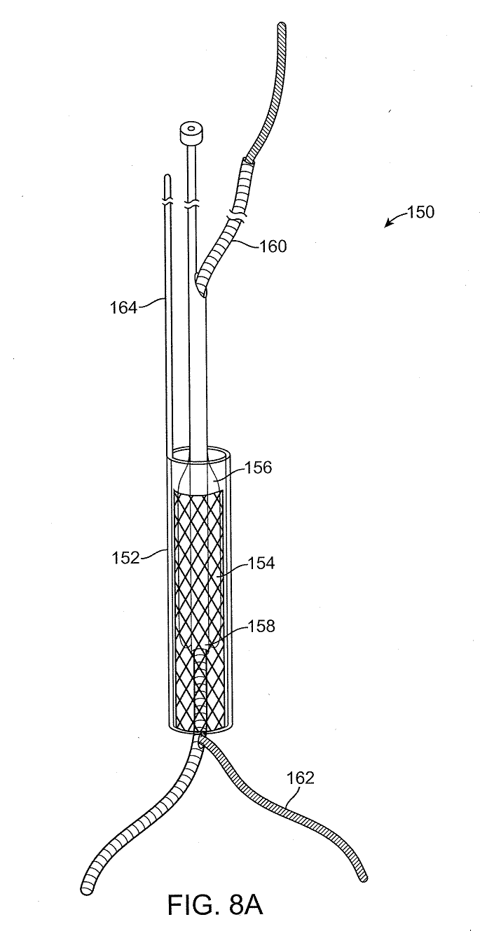

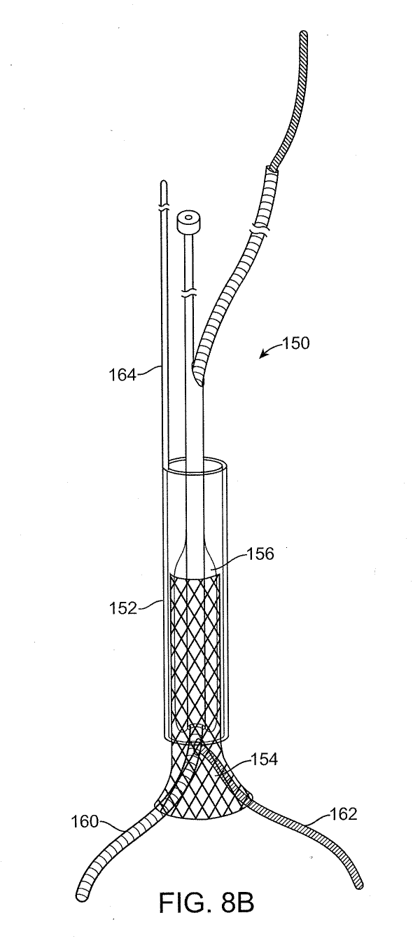

FIGS. 8A and 8B shows a scaffold delivery system including delivery catheter 150 and retractable sheath 152 covering a portion of the catheter, and at least partially covering a scaffold 154 disposed on the catheter. The system is illustrated having a delivery catheter, e.g., similar to embodiment described above with reference to FIG. 5, though various catheter designs may be used. The catheter 150 includes a distally positioned expandable balloon 156 and a guidewire lumen 158, having a distal opening and a proximal opening. A non-bifurcated or cylindrical scaffold 154 is shown having a proximal portion positioned over the balloon 156 and a distal portion that extends beyond the balloon and distally. The catheter can be advanced over a positioned guidewire 160, as described herein. The delivery catheter is shown positioned over a bifurcated guidewire system (although various guidewire configurations may be utilized). The guidewire system includes a first or outer guidewire 160 including a lumen through which a second guidewire 162 (or inner guidewire) can be positioned. The first guidewire 160 includes a distal opening 163 through which the second guidewire 162 can be advanced so as to form a distal guidewire bifurcation. The system further includes an outer sheath 152 positioned over the catheter so as to at least partially cover the scaffold 154. The sheath 152 is coupled to an elongated member 164 that allows control of movement and/or positioning of the sheath during use.

In use, the guidewire system as illustrated in FIGS. 8A and 8B can be positioned, e.g., by advancing the first guidewire through a lumen of the patient and so a distal portion of the first guidewire enters into a branch lumen of a bifurcation. The distal opening of the first guidewire will be positioned proximally, e.g., upstream, of the bifurcation and the second guidewire can be advanced through the lumen of the first guidewire, with a distal portion of the second guidewire advanced out the opening in the wall of the first guidewire and into a second branch lumen of a bifurcation. The catheter delivery system can be advanced over the first guidewire, either prior to or following positioning of the second guidewire as described, or with the second guidewire loaded into the first guidewire lumen. The catheter is positioned proximally or upstream of the bifurcation with the distal portion of the scaffold disposed within the sheath. Once in the desired position, the sheath can be moved or withdrawn proximally so as to expose a distal portion of the scaffold. With a distal portion of the scaffold at least partially exposed outside the sheath, the catheter system can be advanced distally so that the distal portion of the scaffold spreads or flares over the guidewire bifurcation, as shown in FIG. 8B, for positioning the scaffold in the patient's body lumen bifurcation. Once the sheath is withdrawn from covering the balloon, scaffold positioning can be accomplished in a staged manner similar to described further herein. The balloon 156 of the catheter is inflated so as to deploy the proximal portion of the scaffold upstream or proximal to the bifurcation. The catheter can then be withdrawn and a deployment system advanced for fully expanding the distal portion of the scaffold, e.g., as described above.

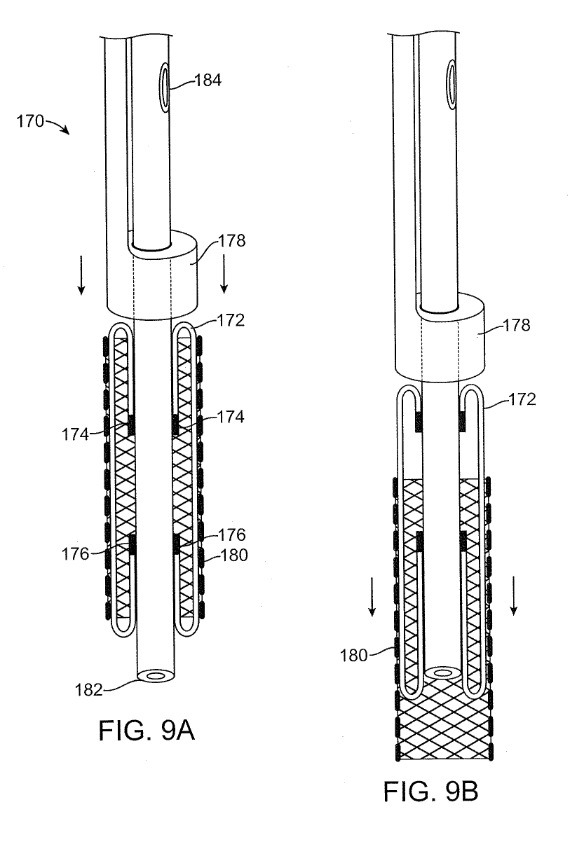

Another embodiment of a scaffold delivery system is described with reference to FIGS. 9A and 9B. The system includes a delivery catheter 170 having an expandable balloon 170 disposed on a distal portion of the catheter, the catheter configured to provide movement of the balloon longitudinally or along the long axis of the catheter. The balloon is coupled to the catheter outer wall at least at points 174, 176, with balloon ends inverted and extending along the catheter length. The balloon is unattached proximal to point 174 as well as distal to point 176 such that a force applied to the balloon allows the balloon to roll along the length of the catheter. For example, a force applied to the proximal portion or end of the balloon can allow the balloon to sort of roll along the catheter length in a distal direction. In one embodiment, as illustrated in FIGS. 9A and 9B, the system includes an elongated member 178 having a distal portion slideably coupled to the shaft of the catheter proximal to the balloon. The elongated member 178 further includes a proximal portion extending proximally and configured to allow application of a force to the distal portion of the member for actuating or sliding the distal portion along the catheter length. A scaffold 180 can be positioned at least partially over the balloon for advancement and positioning in a lumen of a patient. As shown in FIG. 9A, a scaffold can initially be positioned entirely over the balloon, or alternatively can be positioned with less than the entire length of the scaffold disposed over the balloon. Actuation of the member 178 distally along the length of the catheter so as to apply a force about the proximal end of the balloon 172, in turn, allows the balloon 172 and scaffold 180 to roll forward and distally along the catheter length. As the balloon moves distally, the distal portion of scaffold is displaced off the distal portion of the balloon as the balloon inverts or folds inward at the distal end as being secured at anchor or attachment points 174, 176. The positioning of the scaffold and movement of the balloon as described allows placement of the scaffold with respect to the balloon such that the proximal portion of the scaffold is disposed on the balloon, while a distal portion of the scaffold extends distal to the balloon.

In use, a scaffold 180 can be positioned on the balloon 172, e.g., as illustrated in FIG. 9A, and the catheter 170 advanced through a body lumen over a guidewire positioned in the body lumen. Guidewire configurations and positioning can be accomplished as described herein, including use of a single guidewire, a bifurcated guidewire, or a plurality of guidewires. A guidewire(s) is advanced through the body lumen and into position at a bifurcation. The catheter is advanced over the guidewire(s) running through the catheter lumen having openings 182, 184. The catheter is advanced to the bifurcation, and member 178 is actuated to apply a force to the balloon, resulting in the balloon rolling forward or distally, and the scaffold rolling distally to extend further from the distal end of the balloon relative to the starting position of the balloon prior to member actuation, as described above. The actuation of the member and movement of the balloon as described, alone or in combination with movement of the catheter (e.g., withdrawing the catheter shaft proximally), allows the distal portion of the scaffold to be advanced into the bifurcation. The balloon can then be expanded or inflated for expansion and positioning of the proximal portion of the scaffold, or the portion of the scaffold positioned over the balloon following member actuation and balloon movement as described. The catheter can then be withdrawn and a second expansion system (e.g., pair of kissing balloons) advanced to the bifurcation and expanded for expansion and positioning of the distal portion of the scaffold. FIG. 9 depicts a scaffold delivery system with a single guidewire lumen. The same rolling scaffold delivery system, except with two guidewire lumens (not depicted), could be used with two guidewires. The scaffold delivery system could either be advanced to the bifurcation over two guidewires or advanced near to the bifurcation over one guidewire with the second guidewire “loaded” on the catheter, as described previously.

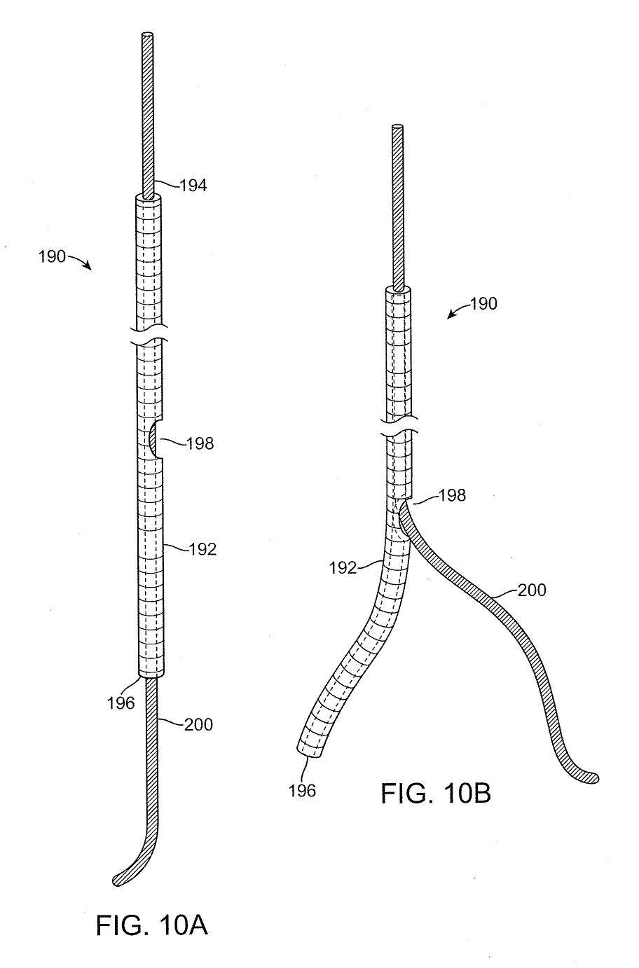

As mentioned above, various guidewire configurations and assemblies can be utilized according to the methods and systems of the present invention. One exemplary guidewire apparatus or combination is described with reference to FIGS. 10A and 10B, where a bifurcated guidewire assembly is illustrated. The assembly 190 includes an outer tube or first guidewire 190 having a proximal portion and distal portion, and further including a lumen extending through a length of the guidewire with the guidewire wall having holes or openings to the lumen including proximal opening 194, distal opening 196, and opening 198 disposed there between and on a side of the tube wall. Typically, opening 198 will be disposed on a distal portion of the first guidewire 192. The assembly system further includes an inner wire or second guidewire 200 sized and configured for advancement through the lumen of the first guidewire 192. In use, in one embodiment, the first 192 and second 200 guidewires can be advanced as one through a body lumen and into a desired position, such as into a first branch lumen of a bifurcation. The first guidewire 192 can then be positioned such that the opening 198 is at or near the bifurcation. The second guidewire 200 is then withdrawn proximally through the first guidewire lumen 192 such that the distal end of the second guidewire is proximal to the opening 198, with positioning of the second guidewire then adjusted or rotated and advanced such that the second guidewire extends out opening 198 and can be advanced into a second branch lumen of a bifurcation. Alternatively, the first guidewire 192 can be first advanced into a desired position with the second guidewire 200 then advanced through the lumen of the first guidewire, or both guidewires can be positioned together, e.g., with the second guidewire “loaded” into the lumen of the first guidewire.

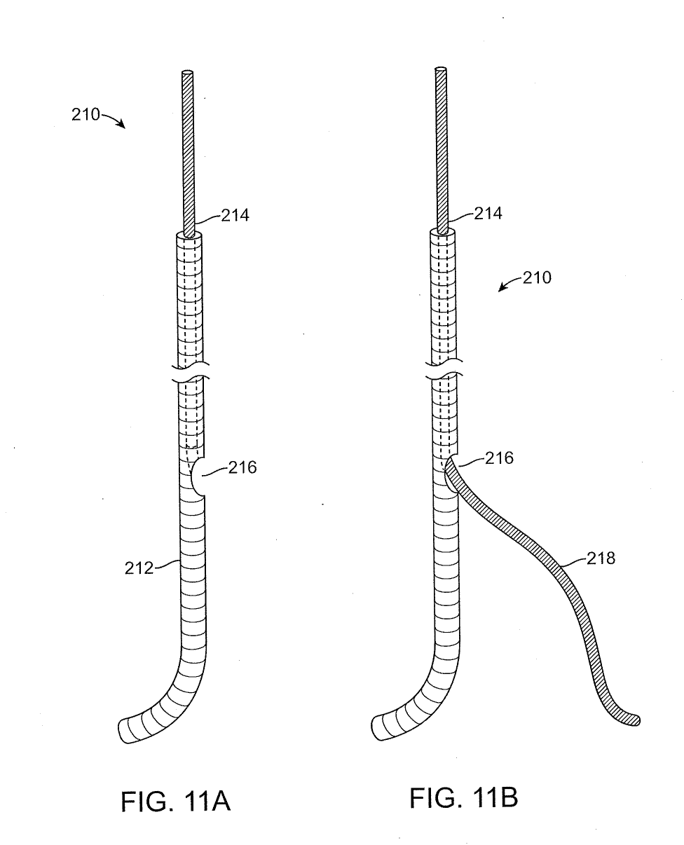

A guidewire apparatus or assembly according to another embodiment of the present invention is described with reference to FIGS. 11A and 11B. The assembly 210 includes an outer tube or first guidewire 212 having a proximal portion and distal portion, and further including tube wall openings 214 and 216, with a lumen extending there between through the body of the guidewire 212. The tube 212 includes an elongated portion extending distal to the opening 216. The assembly 210 further includes an inner wire or second guidewire 218 sized for advancement though the lumen of the outer tube 212.

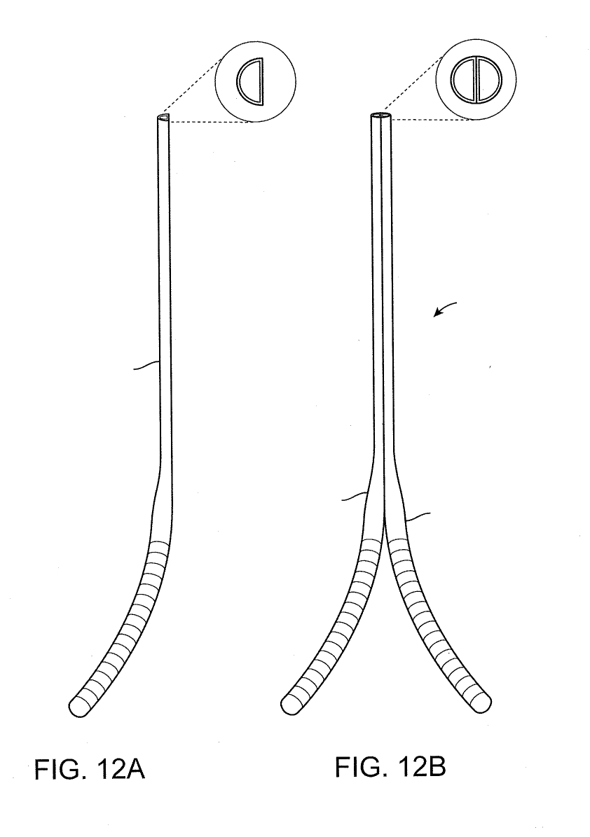

As noted above, a guidewire configuration suitable for use with systems and methods of the present invention can include a multi-guidewire assembly or apparatus. FIGS. 12A and 12B show an exemplary guidewire assembly 220. A single guidewire 222 is shown in FIG. 12A, the guidewire including a proximal portion and a distal portion having different cross-sectional geometries. The proximal portion can include a flattened or planar surface along a length of the wire, such as a “D-shaped” cross-sectional geometry. As shown in FIG. 12B, such a configuration advantageously allows two guidewires 222, 224 to couple together, e.g., along the proximal portions, and may reduce wire tangling or guidewire wrap, reduce unwanted torque during use, and/or allow the distal portion to more easily or controllably assume the desired arrangement. The distal portion of wire 222 or 224 is shown having a generally cylindrical geometry or circular cross-sectional shape.

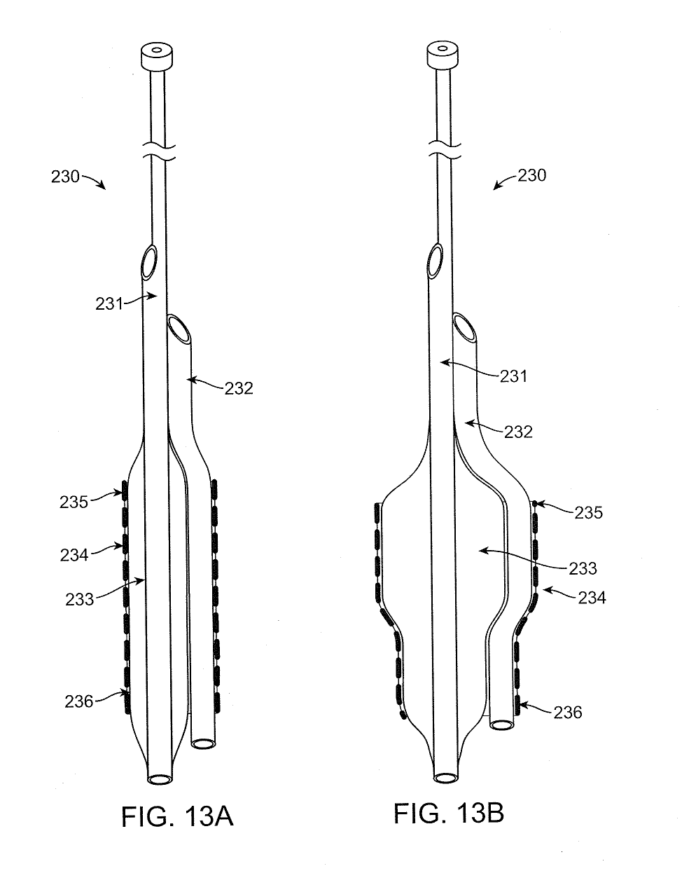

Another embodiment of the scaffold delivery system is depicted in FIGS. 13A and 13B. In FIG. 13A the uninflated delivery system 230 has two guidewire lumens 231, 232 with an inflatable balloon 233 encircling one guidewire lumen 231. The scaffold 234 is positioned such that the proximal end 235 and the distal end 236 are both disposed over the balloon. FIG. 13B depicts the inflated delivery system 230 with guidewire lumens 231 and 232. Inflation of the balloon 233 expands the stent 244. The proximal end of the stent 245 is fully expanded and the distal end 246 partly expanded. The inflated diameter of the proximal portion of the balloon 247 is greater than the inflated diameter of the distal portion of the inflated balloon 248.

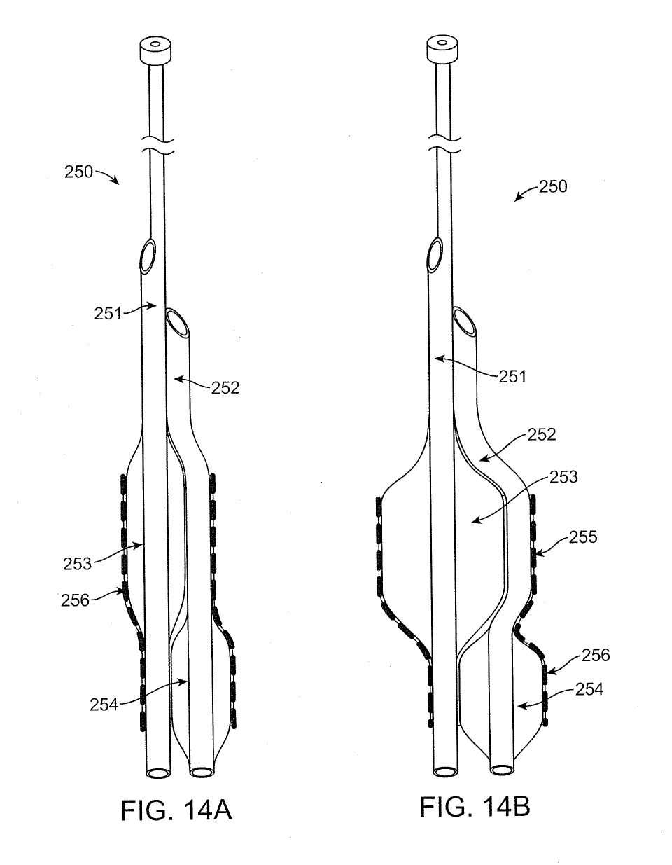

FIGS. 14A and 14B depict another embodiment of the scaffold delivery system. FIG. 14A depicts an uninflated delivery system 250 with two guidewire lumens 251, 252 with an inflatable balloon 253 encircling guidewire lumen 251 and another inflatable balloon 254 encircling guidewire lumen 252. The scaffold 255 is positioned such that the proximal end 256 is disposed over balloon 253 and the distal end 257 is disposed over balloon 254. FIG. 14B depicts the inflated delivery system 250 with guidewire lumens 251 and 252. Inflation of balloons 253 and 254 may be simultaneous or in seriatim. Inflation of balloon 253 expands the proximal portion of the scaffold 255. Inflation of balloon 254 partially expands the distal portion of the scaffold 256. The inflated diameter of balloon 253 is greater than the inflated diameter of balloon 254.

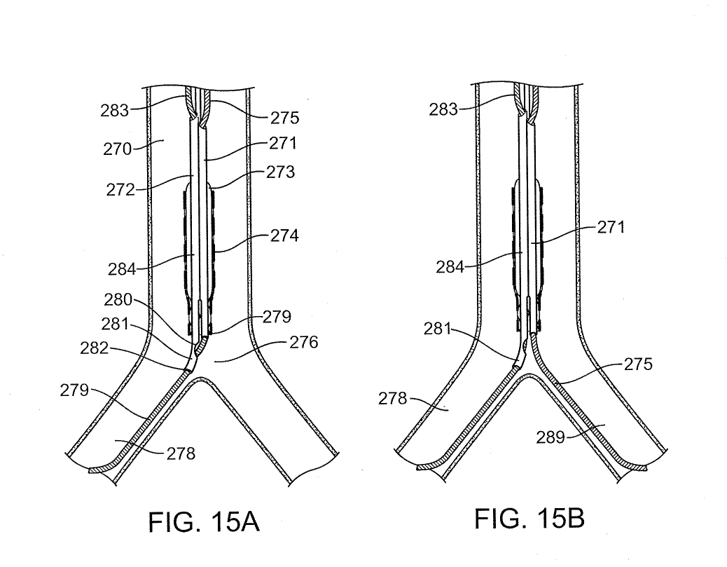

FIGS. 15A and 15B depict the distal portion of another embodiment of a scaffold delivery system with two guidewire lumens. In FIG. 15A the uninflated scaffold delivery system 270 with two guidewire lumens 271, 272 an inflatable balloon 273 and a scaffold 274 is advanced over a first guidewire 275 towards the bifurcation 276. The distal portion of the guidewire 277 is positioned in a first branch vessel 278. The first guidewire 275 traverses a first guidewire lumen 271, exits from its distal end 279, enters a sidehole on a second guidewire lumen 280, traverses a distal portion 281 of the second guidewire lumen 272, and then exits from its distal end 282. The distal portion of a second guidewire 283 is positioned in the proximal portion of the second guidewire lumen 284. The scaffold delivery system 270 and the second guidewire 283 are advanced as one unit towards the bifurcation 276, over the first guidewire 275. In FIG. 15B, the first guidewire 275 is withdrawn from the first branch vessel 278 and from the distal portion of the second guidewire lumen 281 into the first guidewire lumen 271. It is then advanced into the second branch vessel 289. The second guidewire 283 positioned in the proximal portion of the second guidewire lumen 284 is then advanced through the distal portion of the second guidewire lumen 281 and into the first branch vessel 278.

The systems and methods for delivery of a stent or scaffold at a blood vessel or body lumen bifurcation described herein may be used with standard tubular scaffolds or stents. They may also be used with tubular scaffolds or stents with modified features to enhance treatment or repair of the bifurcation. By way of example, aspects of the tubular scaffold or stent which may be modified include the number of circumferential elements, length of circumferential elements, number and configuration of connecting elements, and strut thickness. These modifications may be applied to all of the tubular scaffold or stent, or part of the tubular scaffold or stent, such as the distal portion. These modifications may improve treatment or repair of the bifurcation, for example by extending coverage further into the bifurcation, or increasing the radial force applied to the outer flanks of the bifurcation.

As noted above, a vessel or lumen bifurcation as discussed herein can include a generally symmetric as well as a bifurcation that is generally not symmetric. For illustration purposes, bifurcations are generally shown herein as substantially symmetric. It will be recognized that systems and methods of the present invention will not be limited to any particular anatomical arrangement of a bifurcation, and will include methods and systems configured or suitable for treatment of bifurcations of a wide range of morphological structure or arrangement.

It is understood that the examples and embodiments described herein are for illustrative purposes only and that various modifications or changes in light thereof will be suggested to persons skilled in the art and are included within the spirit and purview of this application and scope of the appended claims. Numerous different combinations are possible and such combinations are considered part of the present invention.

What is claimed is:

1. A method for positioning a scaffold at a bifurcation of a main lumen into first and second branch lumens, said method comprising:
advancing a first balloon catheter system to the bifurcation, the first catheter system having a non-bifurcated scaffold positioned over a main balloon or balloons disposed on a distal portion of the catheter, the scaffold comprising a proximal portion positioned over the balloon(s) and a distal portion positioned either over the balloon(s) or distally of the balloon(s), wherein the balloon catheter system is advanced until the distal portion of the scaffold is aligned adjacent to or approaches the bifurcation;
inflating the main balloon(s) to expand the proximal portion of the scaffold on a generally proximal side of the bifurcation while the distal portion of the scaffold remains either unexpanded or partially expanded;
introducing a second scaffold expansion system having a first balloon and second balloon, the second system advanced so that said first and second balloons are positioned within the distal portion of the scaffold; and
inflating the first and second balloons to expand the distal portion of the scaffold on a generally distal side of the bifurcation.

2. The method of claim 1, wherein the proximal portion of the scaffold and the distal portion of the scaffold are expanded in seriatim.

3. A method for positioning a cylindrical scaffold at a bifurcation of a main lumen into first and second branch lumens, said method comprising:
placing a first guidewire from the main lumen into the first branch lumen and a second guidewire from the main lumen into the second branch lumen;
advancing a main balloon catheter over at least one guidewire, wherein a proximal portion of the scaffold is positioned over the balloon(s) and a distal portion of the scaffold is positioned either over the balloon(s) or distally of the balloon(s), wherein the balloon catheter is advanced until the distal portion of the scaffold has entered the bifurcation;
inflating the main catheter balloon(s) to expand the proximal portion of the scaffold on a generally proximal side of the bifurcation while the distal portion of the scaffold remains either unexpanded or partially expanded, with one guidewire or portion of a guidewire apparatus positioned in the first branch lumen distal to the bifurcation and a second guidewire or portion of a guidewire apparatus positioned in the second branch distal to the bifurcation;
introducing first and second balloon catheters over a first guidewire from the main lumen into the first branch lumen and over a second guidewire from the main lumen into the second branch lumen, respectively, so that said first and second balloons are positioned side-by-side within the distal portion of the scaffold; and
inflating the first and second balloons to expand the distal portion of the scaffold on a generally distal side of the bifurcation.

4. The method of claim 3, wherein the lumens are blood vessels.

5. The method of claim 4, wherein the blood vessels are arteries.

6. The method of claim 3, the catheter comprising at least one lumen extending through a substantially complete length the main balloon catheter to receive a guidewire in an over-the-wire manner.

7. The method of claim 3, the main catheter comprising at least one guidewire lumen extending through only a distal portion of the main balloon catheter to receive a guidewire in a rapid exchange manner.

8. The method of claim 3, wherein advancing the main balloon catheter causes the distal portion of the scaffold to partially open into either or both branch lumens.

9. The method of claim 8, wherein a distal portion of the main balloon catheter is bifurcated with one bifurcation including the first guidewire lumen and following the first guidewire and a second bifurcation, including the second guidewire lumen and following the second guidewire.

10. A scaffold delivery catheter, comprising:
a catheter body having a distal end, a proximal end, and at least one guidewire lumen extending to the distal end, a main inflatable balloon or balloons disposed on the catheter body at a predetermined distance proximally of the distal end; and
a cylindrical scaffold having a proximal portion disposed over the inflatable balloon(s) and a distal portion disposed either over the balloon(s) or distally of the inflated balloon(s), the scaffold disposed over the balloon(s) such that inflation of the inflatable balloon(s) will expand the proximal portion of the cylindrical scaffold while leaving the distal portion of the scaffold either unexpanded or partially expanded.

11. The scaffold delivery catheter of claim 10, the catheter comprising a catheter body having at least one guidewire lumen extending to a non-bifurcated distal end.

12. The scaffold delivery catheter of claim 10, the catheter comprising a catheter body having a distal end, a proximal end, a first guidewire lumen extending to the distal end, and a second guidewire lumen extending to the distal end.

13. The scaffold delivery catheter of claim 10, the catheter comprising a first guidewire lumen and a second guidewire lumen, and an inflatable balloon encircling the first guidewire lumen and the second guidewire lumen.

14. The scaffold delivery catheter of claim 10, the catheter comprising a first guidewire lumen and a second guidewire lumen, and an inflatable balloon encircling the first lumen but not the second lumen.

15. The scaffold delivery catheter of claim 10, further comprising a retractable outer sheath having a first position where the sheath at least partially cover of the cylindrical scaffold, and a second position where the cylindrical scaffold is substantially uncovered by the sheath.

16. The scaffold delivery catheter of claim 10, the catheter comprising at least one guidewire lumen of sufficient caliber to accommodate two guidewires.

17. The scaffold delivery catheter of claim 10, wherein a main inflatable balloon is coupled to the main catheter such that application of a force of sufficient magnitude and direction to the balloon causes advancement of the balloon distally with respect to a longitudinal length of the catheter.

18. The scaffold delivery catheter of claim 17, wherein a main balloon comprises inverted proximal and distal ends.

19. The scaffold delivery catheter of claim 18, wherein a main balloon is attached to the main catheter distal portion at contact points disposed along a length of the main catheter covered by the balloon so as to allow a rolling balloon advancement in a generally distal direction prior to inflation.

20. The scaffold delivery catheter of claim 10, the catheter comprising at least one lumen extending through a substantially complete length of the catheter so as to receive a guidewire in an over-the-wire manner.

21. The scaffold delivery catheter of claim 10, the catheter comprising at least one guidewire lumen extending through only a distal portion of the catheter so as to receive a guidewire in a rapid exchange manner.

22. The scaffold delivery catheter of claim 10, the catheter comprising a bifurcated distal segment distal to the balloon, the distal segment having a bifurcated first guidewire lumen and a second guidewire lumen.

23. The scaffold delivery catheter of claim 10, the balloon having a length in a range from about 4 mm to about 26 mm, the cylindrical scaffold having a length in a range of about 6 mm to about 38 mm, and the distal portion of the cylindrical scaffold disposed distally of the inflated balloon having a length in a range of about 2 mm to about 12 mm.

24. The guidewire assembly suitable for use with the scaffold delivery catheter of claim 10, comprising a first wire having a lumen extending through a length of the first wire, an opening to the lumen at a distal end of the first wire, and at least one opening to the lumen disposed in the first wire body and proximal to the distal end, and a second wire sized for advancement through the lumen of the first wire.

25. The guidewire assembly suitable for use with the scaffold delivery catheter of claim 10, comprising a first wire having a lumen extending through a length of the first wire, an opening to the lumen disposed in the first wire wall and proximal to the distal end, and a substantially solid wire portion extending from the opening to the distal end of the first wire; and a second wire sized for advancement through the lumen of the first wire.

26. The guidewire assembly suitable for use with a scaffold delivery catheter of claim 10, the assembly comprising a first guidewire and a second guidewire, each having a proximal portion and a distal portion, the distal portion having a generally circular cross-sectional geometry and the proximal portion having a D-shaped cross section.

27. A system for delivering a cylindrical scaffold to a bifurcation of a main body lumen into first and second branch lumens, said system comprising:
a scaffold delivery catheter as in claim 8; and
a secondary expansion system, comprising a first balloon and a second balloon, wherein the second expansion system is configured for first and second balloon side-by-side positioning at or near the bifurcation and expansion of the distal portion of the cylindrical scaffold after the proximal portion has been expanded by the main inflatable balloon.

28. The system of claim 27, wherein the secondary expansion system comprises a first catheter having the first balloon and a second catheter having the second balloon.

29. The system of claim 27, wherein the secondary expansion system comprises a catheter having a bifurcated distal portion comprising a first bifurcation having the first balloon, and a second bifurcation having the second balloon.

30. The scaffold delivery catheter of claim 10, wherein a cylindrical scaffold has a proximal and a distal portion disposed over the inflatable balloon, and wherein the proximal portion of the balloon has a larger inflated diameter than the distal portion of the balloon, such that inflation of the inflatable balloon will expand the proximal portion of the cylindrical scaffold and partially expand the distal portion of the cylindrical scaffold.

31. The scaffold delivery catheter of claim 10, wherein a cylindrical scaffold has a proximal and a distal portion, the proximal portion disposed substantially over one balloon and the distal portion substantially over another balloon.

32. The scaffold delivery catheter of claim 31, wherein the proximal balloon has a greater inflated diameter than the distal balloon.

33. The scaffold delivery balloon of claim 31, wherein the balloons are inflated simultaneously.

34. The scaffold delivery balloon of claim 31, wherein the balloons are inflated in seriatim.

35. The scaffold delivery catheter of claim 10 wherein there are two guidewire lumens and the scaffold delivery balloon is advanced over a guidewire in the distal portion of a first guidewire lumen and in a second guidewire lumen.

36. The scaffold delivery catheter of claim 35, wherein a guidewire in a distal portion of a first guidewire lumen and a second guidewire lumen is withdrawn proximal to the distal portion of the first guidewire lumen and readvanced to extend through only the second guidewire lumen
